(19) United States
(12) Patent Application Publication
(10) Pub. No.: US 2013/0289629 A1
(43) Pub. Date: Oct. 31, 2013

(54) Bone Dowel

(71) Applicant: Michael Miller, (US)

(72) Inventor: Michael Miller, (US)

(73) Assignee: Miller Dowel Company, Wilmette, IL (US)

(21) Appl. No.: 13/458,622

(22) Filed: Apr. 27, 2012

(51) Int. Cl. A61B 17/84

(52) U.S. Cl. 606/300

(57) ABSTRACT

A bone dowel is disclosed that may be used for connecting two or more bone components. The bone dowel may have three dowel sections positioned between a base and a tip with a conical upper section. Each bone dowel section may be positioned in axial alignment adjacent at least one other bone dowel section wherein the diameters decrease over the length of the bone dowel from the base to the tip.

### FIELD OF THE INVENTION

This invention pertains in general to a bone fixation device, and more specifically to bone dowels for reconstruction of bone fractures and the like.

### BACKGROUND OF THE INVENTION

Bone dowels are rod-shaped fasteners that may be installed in a bone to hold fragments of fractured bones together during the healing process. Bone dowels and screws have been used to secure and repair bone fragments. Generally bone screws are formed of metal and suffer from disadvantages, such as leaching of the metal and irritation to surrounding tissue and the lack of flexibility in fitting a metal implant to a specific patient. Such conditions can raise the possibility of removal from the bone. Thus there is the need to move away from the use of metal in implants. Forming a dowel of bone or other biocompatible material for use as an implant in mammals can alleviate these problems. A bone composition, particularly a cancellous bone, will be bio-absorbed into the body after a period of time and thus not be present to cause any problems over the long term.

### OBJECTS OF THE INVENTION

The principal object of the present invention is to provide an improved bone dowel having multiple axially aligned sections that decrease in diameter over the longitudinal axial length of the bone dowel.

Another object of the present invention is to provide a novel bone dowel that is easier to insert into a component opening.

Still another object of the present invention is to provide a novel bone dowel that will provide for minimal deformation of the component that it is being inserted into.

Yet another object of the present invention is to provide a novel bone dowel that will provide for alignment of the components that the bone dowel is being inserted into.

A further object of the present invention is to provide a novel bone dowel that is safer to use.

A further object of the present invention is to provide a novel bone dowel which is simple in design and inexpensive to construct, and is durable and rugged in structure, while allowing bioabsorption into the body after the fracture has begun to mend.

Still further objects and advantages will become apparent from a consideration of the ensuing description and drawings of the invention

### SUMMARY OF THE INVENTION

These and other objects are achieved by a bone dowel of the present invention. In one form of the invention, a bone dowel is provided that may be used for connecting two or more components. In one embodiment, the bone dowel may have three dowel sections positioned between a base and a tip with a conical upper section. Each bone dowel section has a diameter and may be positioned in axial alignment adjacent at least one other bone dowel section wherein the diameters decrease over the length of the bone dowel from the base to the tip. Alternate embodiments may have two, four or more sections and may be a two piece structure with an upper section separate from a lower section. The bone dowel of the subject invention may be formed of autograft bone, which, in use, will expand and become tight in the bone opening, thereby causing a friction fit in the bone with compression of the bone in the opening and thereby create more holding power to maintain the two bone parts together.

### BRIEF DESCRIPTION OF THE INVENTION

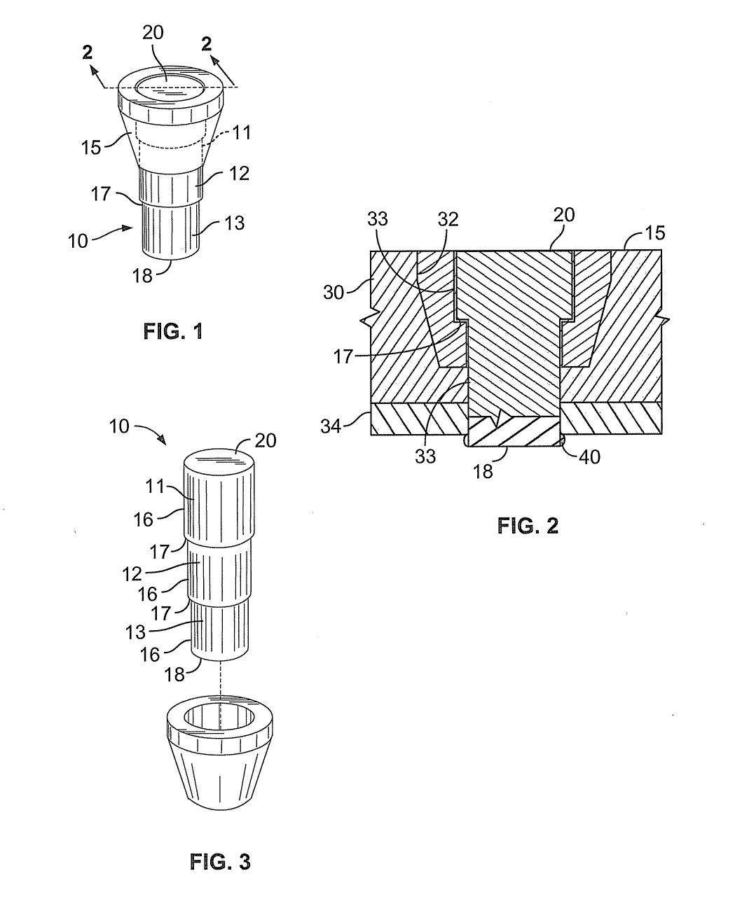

FIG. 1 is a perspective view of one embodiment of the bone dowel of the present invention, showing a two-piece dowel;

FIG. 2 is an cross-section of the bone dowel of FIG. 1 shown connecting two body components;

FIG. 3 is an exploded view of the bone dowel of FIG. 1;

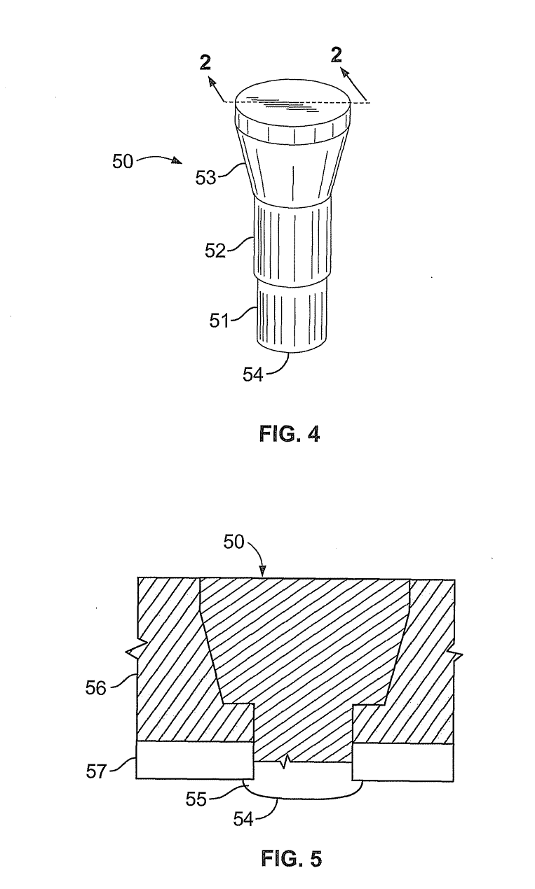

FIG. 4 is a perspective view of a one-piece embodiment of the bone dowel of the subject invention;

FIG. 5 is a cross-section of the bone dowel of FIG. 4, showing connecting two bone components together.

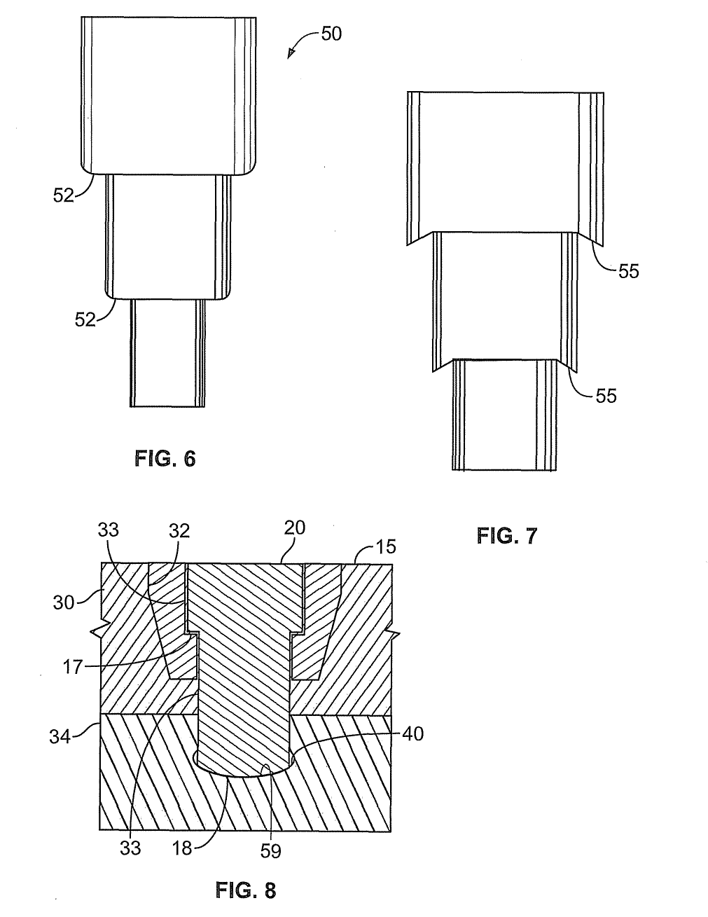

FIG. 6 is an alternate embodiment of the bone dowel of FIG. 4.

FIG. 7 is another alternate embodiment of the bone dowel of FIG. 4.

FIG. 8 is a cross-section of the bone dowel of FIG. 1, showing connecting two bone components together.

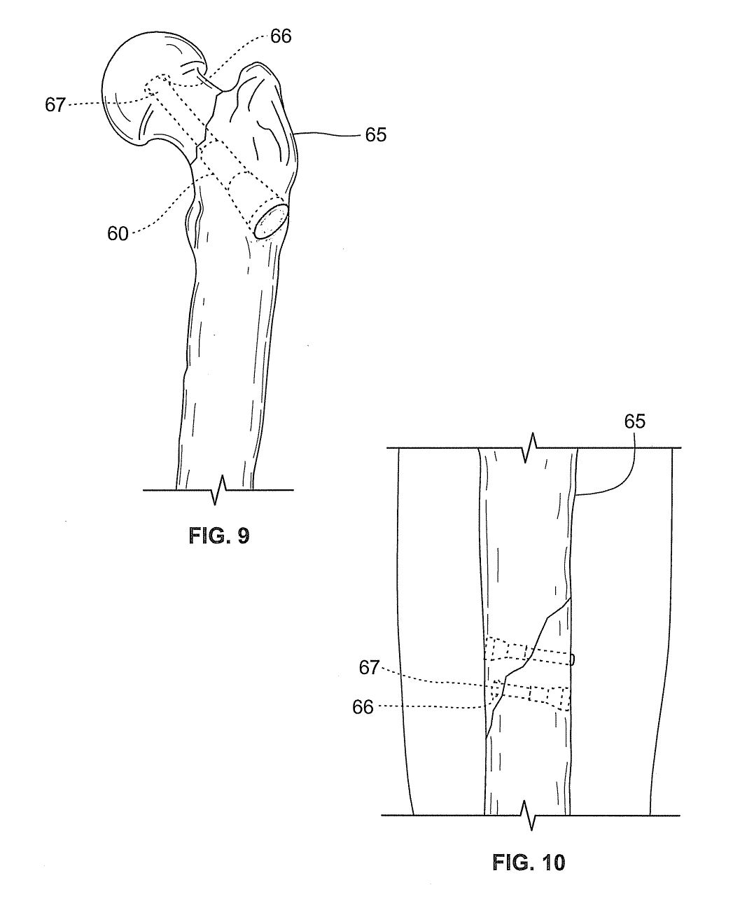

FIG. 9 is perspective view of a femur fracture showing the use of the bone dowel of FIG. 4.

FIG. 10 is a perspective view of a bone fracture showing the use of the bone dowel of FIG. 4.

### DETAILED DESCRIPTION OF THE DRAWINGS

FIG. 1 is a side view of one embodiment of the Bone dowel 10 constructed in accordance with the present invention. FIG. 1 shows one embodiment of the dowel 10 which is comprised of dowel sections 11, 12, 13, each having side walls 16, step portions 17, a tip 18 and a base 20. Each dowel section 11, 12, 13 may have a substantially circular shaped cross section as taken perpendicular to the dowel section 11, 12, 13 longitudinal axis. Other embodiments of the dowel 10 may be constructed so that each dowel section 11, 12, 13 has a substantially square or triangular shaped cross section as taken perpendicular to the dowel section 11, 12, 13 longitudinal axis.

In the embodiment of FIG. 1, the dowel comprises two separate parts, with the upper part 15 being a frustoconical brace 15 in the nature of a washer to assist in the retention of the bone dowel in the bone. The upper part 15 of the dowel has a seat 19 which cooperates with the step portion 17 to stop the further progress of the base 20 through the opening 33.

As shown in FIGS. 1 and 2, lower part 14 has the three dowel sections 11, 12, 13 which may extend from the base 20 and may be longitudinally and axially aligned relative to the base 20 such that the dowel sections 11, 12, 13 are of descending diameter from the base 20 to the tip 18. Depending on the specific application, alternate embodiments of the dowel 10 may include two, four or more dowel sections 11, 12, 13. The individual dowel sections' 11, 12, 13 diameters may be any size and may be joined in any combination required for the particular application that dowel 10 is being used in. The length of each individual dowel section 14 may vary considerably depending on the application and the dimensions of components 30 and 34 (see FIG. 2) that will be joined. In one embodiment, the dowel section 13 having the smallest diameter may have a length that is as long as or longer than the length of any of the other dowel sections 11, 12.

The material selection for the dowel assembly 10 may be dictated by such factors as the location of the bone to be repaired. One embodiment of the dowel 10 may be constructed of cancellous bone, though compact bone is also an option. Bone having collagen content is preferred. In other embodiments, the dowel 10 may be constructed of plastic, metal or any other type of material that will be bioabsorbable and provide for the proper operation of the dowel assembly 10. The dowel 10 may also be constructed of a combination of two or more materials. For example, as shown in FIG. 2, the dowel 10 may be constructed of bone and the brace 15 may be a metal, such as titanium.

As shown in FIG. 1, step portion 17 may be formed by a ninety degree angle with respect to the dowel 10 longitudinal axis. However, depending on the application, the step portion 52 may be configured in a variety of different ways. In alternate embodiments of dowel 10, as shown in FIGS. 6 and 7, step portion 52 may be beveled or convex in shape. Beveled step portion 52 may provide dowel 50 with increased strength to prevent breakage if dowel 50 experiences unusually high tensile or shear forces in a particular application. Also, depending on the type of material dowel 50 is constructed of, the beveled step portion 52 may provide a more secure fit when in use and joining two components 56 and 57. In yet other embodiments, the step portion 55 may be recessed or concave in shape as shown in FIG. 7. In still yet other embodiments, the step portion may be rounded or any other shape that will provide for proper operation of the dowel when used in a particular application. In use, an adhesive such as hydroxyapatite is placed on the side of the bone dowel. The adhesive may be scoured from the dowel when placed into the opening, if the fit is too tight. For this reason, and to accommodate expected expansion of the dowel, the opening is generally sized a small bit, no more than about 0.5 mm, larger in diameter than the dowel. When the dowel is placed into the opening, contact of the walls of the opening with the exterior of the dowel does not take place until the dowel is two thirds of the way into the opening. Thus the adhesive on the sides of the dowel will only scour for a short distance, and then not completely because of the loose fit. It is also possible to place a kerf in the dowel walls extending the length of the dowel, if necessary to relieve the pressure of excess adhesive.

The dowel of the subject invention may be constructed in a variety of different ways using a variety of different manufacturing tools and techniques. For example, dowel 10 may be constructed using a lathe, a tool that is well known in the art. Further, each individual dowel section 11, 12, 13 may be constructed separately and the individual sections may be joined together to form the composite dowel 10. For example, in one embodiment, dowel sections 11, 12, 13 may be fixed together using any type of adhesive or the sections 11, 12, 13 may be fastened together using a fastener such as bone pins or other type of fastener that will provide for the proper operation of dowel 10. Preferably the upper part dowel 14 of FIG. 3 and the dowel 50 of FIG. 4 will each be constructed as a unitary structure. Also the bone dowel of the subject invention may be molded of bioactive polymer, hydroxyapatite or other naturally occurring and biocompatible substances. Currently preferred is autograft bone, although allograft bone, bone graft substitute, such as demineralized bone matrix, hydroxyapatite and bioactive polymers may also be used. An antibiotic or antifungal compound, such as amphotericin B may also be integrally included in the dowel.

Opening 32 may be formed with drill bits as known in the art.

As shown in FIG. 2, during use, force may be exerted on dowel base 20 causing the dowel assembly 10 to be pressed into an opening 32 of first component 30. The opening 32 may be a circular shaped bore with sections 33 that decrease in bore diameter size along the length of opening 32. The second bone component 34 to be connected adjacent the first bone component 30 may be provided with an opening 36 for dowel 10. Adhesive, such as bone putty or the like may be inserted into opening 32, as well as on the bone dowel sections 11, 12, 13 prior to insertion of the dowel into the opening. In addition, the dowel may be coated with the adhesive. When compared with prior art bone dowels with smooth sides, the stepped bone dowel of the subject invention holds the adhesive much better for more efficient and stronger holding power. Once dowel 10 is inserted into first opening 32 and tip 18 is extending out slightly past opening 32, the second bone component opening 36 may be aligned with first bone component opening 32. Dowel tip 18, in combination with bore section area 38, may provide for easy alignment of component openings 32, 36. As a result of the minimal surface area of area 38, tip 18 can easily locate and be aligned with opening 36. Dowel tip 18 is then inserted into opening 36 in second component 34. Pressure may then be applied to the two components 30, 34 to hold them together as dowel 10 is driven into the second component opening 36. As the dowel 10 is inserted into the second component opening 36, the dowel 10 provides for proper alignment of first component 30 and second component 34. As shown in FIG. 3, when dowel 10 is fully in position in openings 32, 36, tip 18 and base 20 of dowel 10 may be substantially flush with the outer surfaces of the components 30, 34 and the first component 30 and the second component 32 will be properly aligned. Multiple bone segments may be secured together by the dowel of the subject invention.

However, in one embodiment, the bone dowel of the subject invention may be inserted into the opening so that it protrudes 1-3 mm from the distal end of the opening 36 as shown in FIG. 2 (exaggerated), where, in time, the bone dowel will hydrate and expand, thereby becoming larger than the opening in that portion, extending beyond the component 34 and forming a self-assembled fastener. Thus, there is preclusion of further movement of the dowel in the opening. Further, since the dowel 10 diameter at that point would be slightly greater in size then the diameters of the corresponding openings sections 33, 36, dowel 10 is held tightly in openings 32, 36 by the frictional forces exerted on the dowel's outer side walls 16 by corresponding openings 32, 36 inner side walls. Thus, when dowel 10 is inserted into the openings 32, 36, steps 17 provide for first component 30 to be held tightly in place adjacent second component 34. In one embodiment each step 17 acts similar to the head of a nail whereby each step 17 prevents the components 30, 34 from sliding off dowel 10 and separating from each other. In the embodiment described above, the opening may extend through the bone and projecting 1-3 mm on the distal end. Thus, when the dowel is set in place and the body is healing, the bone dowel, particularly if formed of cancellous bone with collagen and projecting 1-3 mm on the distal end, will rehydrate and expand, thereby providing a tight fit in the bone and a “cap” 40 at the distal end, comprised of a slight overlaps (shown exaggerated) of the dowel over the edge of opening 33.

In another embodiment of the subject invention, FIG. 4 shows a bone dowel 50 having at least two axially aligned lower sections of decreasing diameter 51 and 52. The upper section 53 is frustoconical, providing a brace or head that will distribute pressure and space the load from the attachment to the bone. As in the embodiment of FIG. 1, the bone dowel of FIG. 5 may be formed of cancellous bone and may be 1-3 mm longer than the bone into which it is driven so that, upon rehydrating, the tip 54 of the bone dowel will expand slightly to extend beyond the opening in which the bone dowel sits and overhang slightly to form a “cap” 55 (shown exaggerated) and thereby tightly secure the bone dowel in its opening.

The dowel of the subject invention may also be used in a situation not allowing or requiring that the dowel go all the way through the bone. In this situation, depicted in FIG. 8, the opening may be drilled so that it has a pocket 59 (shown exaggerated in size) at the bottom of the opening. In this embodiment, the bone dowel will expand into the pocket 59 forming a “cap” 40 and fix the bone dowel in place.

As shown in FIG. 9, the dowel may be used in the case of difficult fractures. Dowel 60 is put in place as described above. In the example of a hip fracture or intracapsular fracture this may be done with a single dowel 60. In cases where the dowel does not go all the way through the bone, a small pocket 67 may be located in the bone at the distal end of the dowel opening to allow for the expansion of the dowel when it rehydrates, thereby forming a “cap” 66.

If desired, the dowel may be supplemented with a plate and screws, with the screws secured to the dowel and in turn securing a plate to the bone. Multiple screws and dowels may be used to secure the plate.

FIG. 10 shows a simple bone fracture held together by dowels 60 alone. The dowels may enter the bone 65 from the same-direction or different directions. A dowel may protrude slightly and expand on the distal end, or not go all the way through and expand into a pocket as previously described.

It will be understood that the foregoing description is of preferred exemplary embodiments of the invention and that the invention is not limited to the specific forms shown or described herein. Various modifications may be made in the design, arrangement, and type of elements disclosed herein, as well as the steps of making and using the invention without departing from the scope of the invention as expressed in the appended claims.

1. A bone dowel having a base and a tip for connecting two bone components, comprising:
a) a lower section comprising at least 3 dowel sections, each dowel section being axially aligned and of successively decreasing diameters;
b) a base section having a frustoconical shape.

2. The bone dowel of claim 1 wherein the base section is separate from the lower section.

3. The bone dowel of claim 1 wherein the bone dowel is formed of cancellous bone.

4. A bone dowel system with a bone dowel for joining bone fragments, said system comprising
a) at least 3 dowel sections, each dowel section being axially aligned and of successively decreasing diameter;
b) a proximal section of frustoconical shape;
c) a distal section;
whereby when in use the done dowel is inserted into a bone opening, so that said distal section extends from said opening, hydrates, and expands at least in circumference of said distal section and thereby expands to form a friction fit in the bone opening.

5. The bone dowel system of claim 4 wherein said distal section is separate from said at least 3 dowel sections.

6. The bone dowel system of claim 4 wherein said bone dowel is formed of cancellous bone having collagen.

7. The bone dowel system of claim 4 wherein said bone dowel extends out of the bone at the distal section.

8. The bone dowel system of claim 7, wherein the distal section of the bone dowel expands beyond the initial diameter of the bone dowel.
